(19) United States
(12) Patent Application Publication
(10) Pub. No.: US 2017/0298400 A1
(43) Pub. Date: Oct. 19, 2017

(54) METHOD FOR PRODUCING PSICOSE

(71) Applicant: INDUSTRY-ACADEMIC COOPERATION FOUNDATION GYEONGSANG NATIONAL UNIVERSITY, Gyeongsangnam-do (KR)

(72) Inventors: Seon-Won KIM, Gyeongsangnam-do (KR); Min-Jin CHOI, Gyeongsangnam-do (KR); Seong-Hee JEONG, Gyeongsangnam-do (KR); Dae-Yun LEE, Gyeongsangnam-do (KR)

(21) Appl. No.: 15/516,342

(22) PCT Filed: Oct. 1, 2015
(86) PCT No.: PCT/KR2015/010407

§ 371 Date: Mar. 31, 2017

(30) Foreign Application Priority Data
Oct. 1, 2014 (KR) 10-2014-0132350

(51) Int. Cl. C12P 19/02; C12P 19/24

(52) U.S. Cl. CPC C12P 19/02; C12P 19/24; C12Y 501/03

(57) ABSTRACT

A method of preparing D-psicose includes a step of reacting D-fructose as a substrate and an epimerase thereof in microorganisms at a temperature of 40° C. or higher. The method may further includes inducing the microorganisms to have resting cells by culturing the microorganisms in a medium not containing the D-fructose before the reaction. The method of preparing D-psicose may significantly improve the production amount of D-psicose and production rate of D-psicose.

### TECHNICAL FIELD

The present invention relates to a method of preparing D-psicose using microorganisms.

### BACKGROUND ART

D-psicose, which is a C-3 epimer of D-fructose, has sweetness similar to common saccharides, but almost zero calories because D-psicose is not metabolized in the body. Thus, D-psicose is a functional saccharide capable of being used as a functional sweetener to replace sugar for diabetic and obese patients. Furthermore, D-psicose has a function of reducing abdominal obesity by inhibiting the activity of enzymes involved in lipid synthesis in the liver, and is currently being studied as a therapeutic agent for diabetes and arteriosclerosis.

Thus, in the food industry, there is a growing need for a method of efficiently preparing D-psicose. Since D-psicose is produced in natural materials in very small amounts during molasses treatment or glucose isomerization, existing methods of preparing D-psicose are mainly chemical processes. Bilik et al. have developed a technique for preparing D-psicose from D-fructose using catalysis of molybdate ions. McDonald prepared D-psicose from 1,2:4,5-Di-O-isopropylidene-beta-D-fructopyranose using a chemical method consisting of a three-step process. In addition, Doner prepared D-psicose by heating D-fructose with ethanol and triethylamine. However, these chemical methods have problems of high production costs, low production efficiency, and excessive production of by-products.

As a method of preparing D-psicose by a biological approach, Ken Izumori et al. demonstrated that microbial cell reactions could be used to prepare D-psicose from D-galactitol, D-tagatose or D-talitol. However, these substrates are costly because the substrates are relatively rare sugars or sugar alcohols in nature.

As an enzymatic conversion method, there is a method of producing D-tagatose-3-epimerase separated from a microorganism, Pseudomonas cichorii ST-24, in recombinant Escherichia coli, purifying the same, and using the D-tagatose-3-epimerase to convert D-fructose into D-psicose. Izumori et al. have prepared D-psicose at a conversion rate of about 25% using a reaction system in which D-tagatose-3-epimerase is immobilized.

As described above, conventionally, to prepare D-psicose from D-fructose, studies have focused on improving the productivity of D-psicose by purifying enzymes and immobilizing the purified enzymes. However, there is a problem in that a process of purifying enzymes requires a lot of time and is costly.

Therefore, there is a need for a method using a strain capable of producing D-psicose with high efficiency and lowering production costs by omitting a process of purifying enzymes.

A method of preparing D-psicose using D-psicose epimerase is disclosed in Korean Patent Application Publication No.2006-125971.

### PRIOR ART DOCUMENTS

### Patent Document

Korean Patent Application Publication No. 2006-125971

### Non-Patent Document

Choi J G, Ju Y H, Yeom S J and Oh D K (2011), IMPROVEMENT IN THE THERMOSTABILITY OF D-PSICOSE 3-EPIMERASE FROM AGROBACTERIUM TUMEFACIENS BY RANDOM AND SITE-DIRECTED MUTAGENESIS, Appl Environ Microbiol 77(20):7316-20.

### DISCLOSURE

### Technical Problem

It is an object of the present invention to provide a method for preparing D-psicose, which may remarkably improve the production amount and the production rate of D-psicose.

### Technical Solution

One aspect of the present invention provides a method of preparing D-psicose, including a step of reacting D-fructose as a substrate and an epimerase thereof in microorganisms at a temperature of 40° C. or higher.

The present inventors developed the present invention by discovering that the production amount and production rate of D-psicose significantly increased when the reaction of D-fructose as a substrate and an epimerase thereof was performed at a temperature of 40° C. or higher and that the production amount and production rate of D-psicose increased as temperature increased.

In general, enzymes such as epimerases are thermally denatured at temperature above 40° C. and lose activity thereof. However, the reaction using an epimerase according to the present invention is performed in microorganisms. Thus, since epimerase is protected in the microorganisms as compared with the case where the epimerase is directly exposed to the outside, the reaction may be performed without denaturing the epimerase even at a high temperature.

As the reaction temperature increases, D-psicose productivity further increases. Accordingly, the reaction temperature is 40° C. or higher, and is not particularly limited as long as the microorganisms are not damaged by heat, and proteins or saccharides are not denatured by heat. For example, the reaction temperature may be 40 to 50° C., 40 to 60° C., 40 to 70° C., 40 to 80° C., 40 to 90° C., 45 to 60° C., 45 to 70° C., 45 to 80° C., 45 to 90° C., 50 to 70° C., 50 to 80° C., 50 to 90° C., 55 to 60° C., 55 to 70° C., 55 to 80° C., 55 to 90° C., 60 to 70° C., 60 to 80° C., 60 to 90° C., 70 to 80° C., 70 to 90° C., and the like. From the viewpoint of maximizing D-psicose productivity, the lower limit of the reaction temperature is preferably 50° C. or higher, whereas the upper limit is preferably 90° C. or lower in view of preventing heat damage and denaturation.

As used herein, “microorganisms” may be cells that are capable of being cultured in a liquid medium.

The microorganisms may express an epimerase endogenously or by transformation. When an epimerase is expressed in the microorganisms, D-psicose generated by the reaction of D-fructose and the epimerase may be continuously produced in the microorganisms.

When an epimerase is expressed in microorganisms transformed with a gene encoding the epimerase, the gene encoding the epimerase may be a gene encoding Agrobacterium tumefaciens-derived D-psicose 3-epimerase corresponding to SEQ ID NO: 1 or a gene encoding Anaerostipes caccae-derived D-psicose 3-epimerase corresponding to SEQ ID NO: 2.

Agrobacterium tumefaciens-derived D-psicose 3-epimerase may have an amino acid sequence of SEQ ID NO: 3, and Anaerostipes caccae-derived D-psicose 3-epimerase may have an amino acid sequence of SEQ ID NO: 4.

From the viewpoint that an epimerase has excellent high-temperature stability, a gene encoding an epimerase is preferably a gene encoding an amino acid sequence of D-psicose 3-epimerase corresponding to SEQ ID NO: 5.

The amino acid sequence of SEQ ID NO: 5 is a sequence in which the 33rd amino acid is substituted with leucine and the 213th amino acid is substituted with cysteine in an amino acid sequence of Agrobacterium tumefaciens-derived D-psicose 3-epimerase, and is described in Reference 1 as having excellent thermal stability.

The present inventors confirmed that Clostridium-derived D-psicose 3-epimerase exhibited excellent thermal stability. Referring to FIG. 7, it was confirmed that Clostridium-derived D-psicose 3-epimerase having high thermal stability had a sequence corresponding to the amino acid sequence with one amino acid substitution or a sequence corresponding to the amino acid sequence with two amino acid substitutions as described above.

Thus, in addition to Agrobacterium tumefaciens-derived D-psicose 3-epimerase, it was confirmed that amino acids corresponding to the 33rd and 213th amino acids of the amino acid sequence of the Agrobacterium tumefaciens-derived D-psicose 3-epimerase were important for increasing thermal stability in D-psicose 3-epimerase derived from other strains.

Specific examples of such amino acid sequences may be a sequence in which the 32nd amino acid is substituted with leucine or the 196th amino acid is substituted with cysteine in an amino acid sequence of SEQ ID NO: 6. SEQ ID NO: 6 corresponds to a sequence represented by the boxes in FIG. 7, and is a common base sequence of Agrobacterium tumefaciens-, Anaerostipes caccae-, Clostridium bolteae-, and Clostridium hylemonae-derived D-psicose 3-epimerase amino acid sequences (SEQ ID NO: 3, 4, 9, and 10) used herein. Therefore, the microorganisms may be transformed with genes encoding the amino acid sequences, without being limited thereto.

In addition, from the viewpoint of excellent high-temperature stability and D-psicose productivity, an epimerase may be Clostridium-derived D-psicose 3-epimerase, and a gene encoding the same may be a gene encoding Clostridium bolteae-derived D-psicose 3-epimerase corresponding to SEQ ID NO: 7, or a gene encoding Clostridium hylemonae-derived D-psicose 3-epimerase corresponding to SEQ ID NO: 8. From the view point of maximizing high-temperature stability, the gene is preferably a gene encoding Clostridium hylemonae-derived D-psicose 3-epimerase corresponding to SEQ ID NO: 8.

Clostridium bolteae-derived D-psicose 3-epimerase may have an amino acid sequence of SEQ ID NO: 9, and Clostridium hylemonae-derived D-psicose 3-epimerase may have an amino acid sequence of SEQ ID NO: 10.

In the case of Anaerostipes caccae-derived D-psicose 3-epimerase, D-psicose 3-epimerase having a sequence in which the 32nd amino acid is substituted with leucine or the 196th amino acid is substituted with cysteine in an amino acid sequence of SEQ ID NO: 6, Clostridium bolteae-derived D-psicose 3-epimerase, and Clostridium hylemonae-derived D-psicose 3-epimerase among the above-described D-psicose 3-epimerases, pH at which the D-psicose 3-epimerases exhibit optimal activity is as low as 7 or less.

The microorganisms may be prokaryotic or eukaryotic cells, may be cultured in liquid media, and may be cultured at the above-described high temperature. For example, the microorganisms may be bacteria, fungi, or combinations thereof. The bacteria may be gram-positive bacteria, gram-negative bacteria, or combinations thereof. From the viewpoint of increasing D-psicose productivity, the bacteria are preferably gram-positive bacteria. The gram-negative bacteria may be Escherichia. The gram-positive bacteria may be Bacillus, Corynebacterium, Actinomyces, lactic acid bacteria or combinations thereof. The fungi may be yeasts, Kluyveromyces, or combinations thereof.

In a method of preparing D-psicose according to the present invention, since the reaction of D-fructose and an epimerase is performed at a temperature of 40° C. or higher, thermophiles having high thermal stability are preferable as the microorganism. For example, the thermophiles may be Corynebacterium and Actinomyces, more preferably Corynebacterium glutamicum, most preferably Corynebacterium glutamicum in which the above-described gene encoding an epimerase is introduced into Corynebacterium glutamicum ATCC 13032.

The Escherichia microorganisms may be Escherichia coli, specifically DH5α, MG1655, BL21(DE), S17-1, XL1-Blue, BW25113 or combinations thereof, into which a gene encoding an epimerase is introduced.

In addition, the Escherichia coli may be one in which one DNA region consisting of a gene encoding endogenous 6-phosphofructokinase and an operon responsible for allose metabolism is inactivated.

For example, the gene encoding 6-phosphofructokinase may have a nucleotide sequence of SEQ ID NO: 11 and the 6-phosphofructokinase may have an amino acid sequence of SEQ ID NO: 12.

Genes consisting of an operon responsible for allose metabolism include rpiB, alsR, alsB, alsA, alsC, alsE, and alsK and one or more thereof may be inactivated.

For example, rpiB, alsR, alsB, alsA, alsC, alsE, and alsK genes may correspond to nucleotide sequences of SEQ ID NO: 13, 14, 15, 16, 17, 18 and 19, respectively.

RpiB, alsR, alsB, alsA, alsC, alsE, and alsK genes may encode amino acid sequences of SEQ ID NO: 20, 21, 22, 23, 24, 25 and 26, respectively.

The term “inactivation” indicates that expression of the genes is reduced or the genes are not expressed. “Inactivation” may be achieved by methods known in the art. For example, the genes may be inactivated by homologous recombination. For example, the homologous recombination may be mediated by transposon mutagenesis or P1 transduction.

Corynebacterium microorganisms may be Corynebacterium glutamicum, specifically Corynebacterium glutamicum in which a gene encoding an epimerase is introduced into Corynebacterium glutamicum ATCC 13032.

Corynebacterium microorganisms may have a defective or inactivated ptsF gene (EII^{Fru}, fruA, NCg11861, GI:19553141, EC 2.7.1.69) responsible for the PTS transport system that converts endogenous di-fructose into di-fructose-1-phosphate and transports di-fructose-1-phosphate into microorganisms.

The PtsF gene may have a nucleotide sequence of SEQ ID NO: 27, and may encode an amino acid sequence of SEQ ID NO: 28.

Considering that D-psicose is produced from D-fructose, the deficiency or inactivation of the gene may remarkably improve D-psicose production efficiency because phosphorylation of D-fructose is inhibited.

In addition, Corynebacterium microorganisms may have a defective or inactivated mt1D gene (NCg10108, GI:19551360, EC 1.1.1.67) encoding mannitol 2-dehydrogenase.

The mt1D gene may have a nucleotide sequence of SEQ ID NO: 29, and may encode an amino acid sequence of SEQ ID NO: 30.

The reaction of D-fructose as a substrate and an epimerase thereof is performed in microorganisms, and thus microorganisms may be cultured in a medium containing D-fructose.

The medium may be a nutrient medium containing yeast extracts and nitrogen sources such as 2YT, LB, and TB media.

The concentration of D-fructose contained in the medium is not particularly limited. For example, the concentration may be 1 to 80% (w/v), specifically, within this range, the concentration may be 1 to 35% (w/v), 10 to 80% (w/v), 20 to 80% (w/v), 30 to 80% (w/v), 40 to 80% (w/v) and the like. Preferably, the concentration may be 1 to 50% (w/v).

In addition, the medium may be a defined medium commonly used in the art, containing carbon sources including glucose, glycerol and the like; nitrogen sources including ammonia, urea, and the like; essential metal ions including sodium, potassium, calcium, magnesium, manganese, cobalt, and the like; vitamins and the like.

The culture be a continuous, semi-continuous, or batch type culture.

The microorganisms may be inoculated into a medium containing D-fructose such that the turbidity of the microorganisms (absorbance value measured at 600 nm, hereinafter referred to as OD600) is 0.01 to 300. For example, the turbidity may be 1 to 300, 10 to 300, 20 to 300, 5 to 300, or 40 to 300. By using the microorganisms containing such a high concentration of the enzyme, it is possible to efficiently convert D-fructose into D-psicose in a medium containing D-fructose at a high concentration.

The culture may be performed by further adding substances that induce the expression of a gene encoding an epimerase.

The substances inducing gene expression are not particularly limited, and may be substances ordinarily used in the art.

In a method of preparing D-psicose according to the present invention, the reaction of D-fructose and an epimerase may be performed in a medium containing only D-fructose as a substrate, and inorganic salts for providing cofactors. For example, the inorganic salts may be manganese salts or cobalt salts. For an improved production rate of D-psicose, cobalt salts are preferred, and for safe use of produced D-psicose in foods, etc., manganese salts are preferred.

The medium containing only D-fructose and inorganic salts may be a liquid medium in which D-fructose and inorganic salts are dissolved in a solvent. For example, the solvent may be water.

When D-psicose is produced using microorganisms, metabolites such as organic acids of microorganisms other than D-psicose are generated in a medium and the medium may be gradually acidified. The medium containing only D-fructose and inorganic salts according to the present invention does not include a buffer solution. Thus, when the medium is acidified, it is preferable to use D-psicose 3-epimerase having optimum activity at a low pH (e.g., pH 7 or less).

Cultures of the microorganisms include D-psicose. Recovery of D-psicose is not limited to any particular method, and may be performed by methods known in the art. For example, centrifugation, filtration, crystallization, ion exchange chromatography and the like may be used.

Specifically, the culture may be subjected to centrifugation to separate a culture supernatant from microorganisms, and then D-psicose may be recovered from the separated culture supernatant using a recovery method.

The method of preparing D-psicose according to the present invention may further include a step of inducing the microorganisms to have resting cells by culturing the microorganisms in a medium containing no D-fructose before the reaction of D-fructose and an epimerase.

The step of inducing into resting cells may be performed by culturing the microorganisms to a stationary phase in a medium containing no D-fructose.

As used herein, “resting cells” refers to cultured cells that are no longer proliferating. The stationary phase refers to a state in which cell division and proliferation stop after an exponential phase during cell culture and cell population does not increase, and synthesis and decomposition of cellular components are balanced.

Therefore, the resting cells according to the present invention refer to cells in which growth is completed and an epimerase is sufficiently expressed in the cells. When microorganisms are induced to have resting cells, the expression level of an epimerase is maximized, and thus production of D-psicose may be maximized.

The medium containing no D-fructose may be the same as the above-described medium containing D-fructose except that the medium does not contain D-fructose.

In addition, the present invention may further include a step of recovering and reusing the microorganisms to convert another substrate into D-psicose after reacting D-fructose as a substrate and an epimerase thereof.

According to the present invention, the reaction of D-fructose as a substrate and an epimerase thereof is performed in microorganisms. Since epimerase is protected in the microorganisms during the reaction, the epimerase retains enzymatic activity even at a high temperature. Thus, the epimerase may be reused.

That is, the microorganisms may be recovered and reused to convert another substrate into D-psicose after the reaction.

When the reaction is performed in an environment in which growth of separated microorganisms is maintained, the number of times of reuse is not limited, and the microorganisms may be reused hundreds of times or thousands of times.

When the step of reusing microorganisms is further included, since microorganisms are exposed to a high temperature a plurality of times, it is preferable to use thermophiles having high thermal stability in view of high enzymatic activity upon reuse.

Among the above-described embodiments, the microorganisms are preferably Corynebacterium and Actinomyces, more preferably Corynebacterium glutamicum, most preferably Corynebacterium glutamicum in which the gene encoding epimerase described above is introduced into Corynebacterium glutamicum ATCC 13032.

### Advantageous Effects

According to the present invention, the production amount and production rate of D-psicose can be remarkably improved.

According to the present invention, since microorganisms can be recovered and the recovered microorganisms can be reused repeatedly in the course of converting D-fructose to D-psicose, a process yield can be remarkably improved.

### DESCRIPTION OF DRAWINGS

FIG. 1 shows the amount of D-psicose produced from a substrate, D-fructose, depending on temperature in a reaction of converting D-psicose 3-epimerase-introduced Corynebacterium glutamicum transformants into resting cells (i.e., a reaction that produces D-psicose by reacting D-fructose and an epimerase).

FIG. 2 shows the amount of D-psicose produced from a substrate, D-fructose, depending on temperature in a reaction of converting D-psicose 3-epimerase-introduced Escherichia coli MG1655 transformants into resting cells.

FIG. 3 shows the amount of D-psicose produced in reused microorganisms. A reaction that converts transformants of D-psicose 3-epimerase-introduced Corynebacterium glutamicum and D-psicose 3-epimerase-introduced Escherichia coli MG1655 into resting cells was performed at 60° C. for 3 hours, and then the transformants were recovered and reacted under the same reaction conditions to determine the amount of produced D-psicose.

FIG. 4 shows the amount of D-psicose produced from D-fructose depending on the composition of a medium for D-psicose production used in a reaction of converting D-psicose 3-epimerase-introduced Corynebacterium glutamicum transformants into resting cells.

FIG. 5 shows the production amount of D-psicose depending on strains from which D-psicose 3-epimerase is derived and the composition of a medium for D-psicose production when performing a reaction of converting D-psicose 3-epimerase-introduced Corynebacterium glutamicum transformants into resting cells.

FIG. 6 shows the production amount of D-psicose depending on strains from which D-psicose 3-epimerase is derived and heating time when performing a reaction of converting D-psicose 3-epimerase-introduced Corynebacterium glutamicum transformants into resting cells.

FIG. 7 is a result of comparing the amino acid sequences of D-psicose 3-epimerases derived from various strains.

FIG. 8 shows the production amount of D-psicose depending on the number of times of reuse of Corynebacterium glutamicum into which Clostridium-derived D-psicose 3-epimerase was introduced.

### MODES OF THE INVENTION

Hereinafter, the present invention is described in detail with reference to examples.

### EXAMPLES

1. Changes in D-Psicose Production Rate and Production Amount Depending on Temperature in Process of Producing D-Psicose from D-Fructose Using Corynebacterium glutamicum Strain

### (1) Preparation of Recombinant Strains

pCES208 (J. Microbiol. Biotechnol., 18:639-647, 2008), a shuttle vector for Escherichia coli-Corynebacterium, was modified to produce a pSGT208 shuttle vector in which a terminator and a lac promoter were inserted.

To produce D-psicose in Corynebacterium glutamicum, the dpe gene (AGR_L_260, GI:15890243, SEQ ID NO: 1) of Agrobacterium tumefaciens (Agrobacterium tumefaciens str. C58; taxid: 176299; GenBank NID: NC_003062, ATCC33970), which encodes D-psicose 3-epimerase, was introduced into the prepared pSGT208 shuttle vector.

Specifically, a dpe gene was amplified from the genome of Agrobacterium tumefaciens using primer 1 of SEQ ID NO: 31 and primer 2 of SEQ ID NO: 32, digested with restriction enzymes, KpnI and BamHI, and inserted into the pSGT208 shuttle vector digested with the same enzymes to produce a pS208-dpe recombinant shuttle vector containing D-psicose 3-epimerase.

Thereafter, to increase the expression level of D-psicose 3-epimerase in Corynebacterium glutamicum, the lac promoter of the pS208-dpe vector was substituted with a pTrc99a-derived trc promoter. This was named pS208cT-dpe.

The prepared recombinant vectors, pS208-dpe and pS208cT-dpe, containing D-psicose 3-epimerase and pSGT208 vector as a negative control thereof, were introduced into wild-type Corynebacterium glutamicum ATCC 13032, and the transformed Corynebacterium glutamicum ATCC 13032 was used to produce D-psicose from D-fructose. A transformation method followed a method specified in Handbook of Corynebacterium glutamicum (Lothar Eggeling et. al., ISBN 0-8493-1821-1, 2005 by CRC press).

### (2) Cultivation of Recombinant Strains and Production of D-Psicose Using the Same.

To obtain microorganisms at a high concentration, the above-prepared Corynebacterium glutamicum transformants were inoculated into 5 ml of a LB medium (Difco) containing 20 μg/mL of kanamycin and subjected to seed culture at 30° C. and 250 rpm. After culture, the seed culture was inoculated into a minimal medium (1 g K₂HPO₄, 10 g (NH₄)₂SO₄, 0.4 g MgSO₄7H₂O, 20 mg FeSO₄7H₂O, 20 mg MnSO₄5H2O, 50 mg NaCl, 2 g urea, 0.1 mg biotin, and 0.1 mg thiamine per liter) containing 10 g/L of glucose and 20 μg/mL of kanamycin and then subjected to main culture. The main culture was performed in a grooved 500 ml Erlenmeyer flask with a 100 ml volume at 30° C. and 180 rpm for 12 hours to induce sufficient cell mass and sufficient expression of proteins.

The obtained culture solution was centrifuged to remove a supernatant and recover the microorganisms. The microorganisms were resuspended to an OD₆₀₀ value of 40 in the same minimal medium as above described, containing 40% (w/v) D-fructose as a substrate, and then a reaction that converts the microorganisms into resting cells was performed at 25, 30, 37, 50, 60 or 70° C. and 180 rpm.

High-performance liquid chromatography (HPLC) was used to determine the concentrations of D-fructose and D-psicose. HPLC was performed using SCL-10A (Shimadzu, Japan) equipped with a Kromasil 5NH₂ column (4.6 mm×250 mm), and mobile phases were separated at 40° C. with 75% acetonitrile at a flow rate of 1.5 mL/min and analyzed using a RI (Reflective Index) detector. Under the above conditions, retention times were 5.5 minutes for D-fructose and 4.6 minutes for D-psicose.

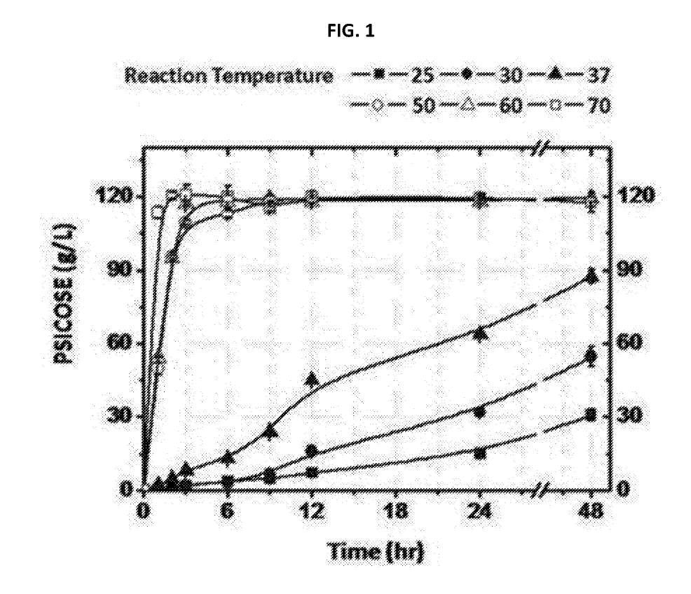

The measurement results are shown in FIG. 1. Referring to FIG. 1, when a conversion reaction was performed on a Corynebacterium glutamicum ATCC13032 strain, in which a pSGT208cT-dpe shuttle vector was introduced, in a medium containing 40% D-fructose, the production rate of D-psicose was remarkably increased and the production amount of D-psicose was increased in proportion to reaction temperature. In particular, in experiment groups reacted at 50, 60, and 70° C., the enzymatic reaction of D-psicose 3-epimerase reached equilibrium within about 3 hours, producing 120 g/L of D-psicose. These results indicated that a conversion rate at which D-psicose 3-epimerase converts D-fructose to D-psicose and the production amount of D-psicose are temperature-dependent.

The production amount of D-psicose increased rapidly from 50° C., which was significantly higher than the temperature required for conventional enzymatic reactions. It is considered that the reaction between the enzyme and the substrate has changed at relevant temperatures.

2. Changes in D-Psicose Production Rate and Production Amount Depending on Temperature in Process of Producing D-Psicose from D-Fructose using Escherichia Coli

According to a method described in Example 1 of Korean Patent Registration No. 10-1106253, a pTPE plasmid was prepared by introducing Agrobacterium tumefaciens-derived D-psicose 3-epimerase into a pTrc99A vector, and an E. coli MG1655(ApfkA, als2) strain was transformed with the pTPE plasmid.

To block the degradation pathway of D-psicose, Escherichia coli MG1655 deficient in pfkA (SEQ ID NO: 11) and als2 (SEQ ID NO: 14, 15, 16, 17, 18 and 19) genes were used.

To obtain microorganisms at a high concentration, the above-prepared Escherichia coli MG1655 transformants were inoculated into 5 ml of a LB medium (Difco) containing 100 μg/mL of ampicillin and subjected to seed culture at 37° C. and 250 rpm. After culture, the seed culture was inoculated into a 2YT medium containing 10 g/L of glucose and 100 μg/mL of ampicillin and then subjected to main culture. The main culture was performed in a grooved 500 ml Erlenmeyer flask with a 100 ml volume at 37° C. and 180 rpm for 12 hours to induce sufficient cell mass and sufficient expression of proteins.

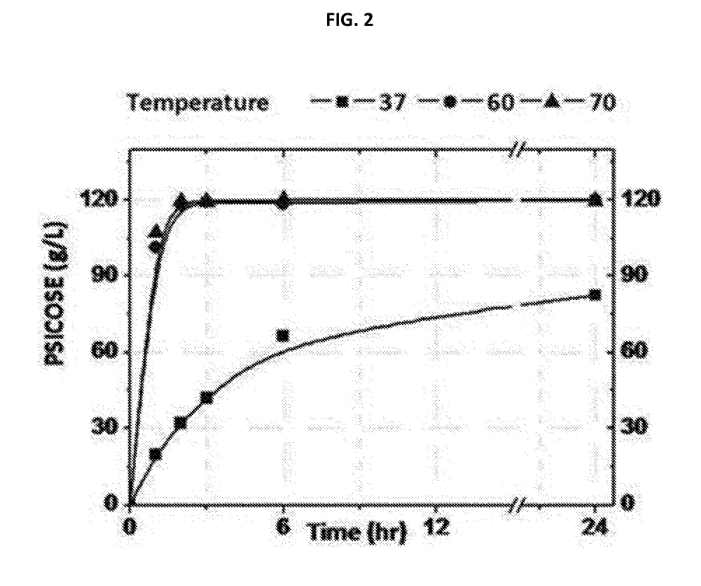

The obtained culture solution was centrifuged to remove a supernatant and recover the microorganisms. The microorganisms were resuspended to an OD₆₀₀ value of 40 in an Escherichia coli minimal medium, a M9 medium (11.3 g M9 minimal salts (Difco), 0.1 mL 1 M CaCl₂, 2 mL 1 M MgSO₄, 1 mL 100 mM MnSO₄5H₂O per liter), containing 40% (w/v) D-fructose as a substrate, and then a reaction that converts the microorganisms into resting cells was performed at 37, 60 or 70° C. and 180 rpm. The concentrations of D-fructose and D-psicose were analyzed according to the method described in Example 1. Measurement results are shown in FIG. 2.

Referring to FIG. 2, when a conversion reaction was performed on an Escherichia coli MG1655 (ApfkA, als2) strain, in which a pTPE vector was introduced, in a medium containing 40% D-fructose, the production rate and production amount of D-psicose were increased in proportion to reaction temperature. In particular, in experiment groups reacted at 60 and 70° C., as with experiments using Corynebacterium, the enzymatic reaction of D-psicose 3-epimerase reached equilibrium within about 2 hours, producing 120 g/L of D-psicose.

These results indicated that a conversion rate at which D-psicose 3-epimerase converts D-fructose to D-psicose and the production amount of D-psicose are temperature-dependent.

When a reaction of converting microorganisms into resting cells was performed at a high temperature on representative gram-positive bacteria, Corynebacterium, and representative gram-negative bacteria, Escherichia coli, since D-psicose 3-epimerase, a glycosyltransferase, was protected from an extreme external environment in cells, the D-psicose 3-epimerase produced D-psicose at a higher rate without heat denaturation at high temperature in comparison with a pure enzyme state. The advantage of such cell conversion reaction is that the reaction may be applied to most microorganisms.

3. Continuous Production of D-Psicose from D-Fructose by Recovering and Reusing Cells in Reaction of Converting Corynebacterium glutamicum Transformants into Resting Cells

In the results of Examples 1 and 2, when D-psicose 3-epimerase was introduced into Corynebacterium and Escherichia coli transformants and a conversion reaction was performed at a high temperature of 50° C. or more, the production amount of D-psicose reached a maximum within 3 hours and did not increase anymore.

To confirm whether D-psicose 3-epimerase still retained the activity of converting D-fructose into D-psicose even after 3 hours at which the reaction equilibrium and maximal production of D-psicose were reached, the microorganisms, which were used for D-psicose production in the conversion reaction for 3 hours in the presence of D-fructose, were recovered and reused in another conversion reaction for D-psicose production.

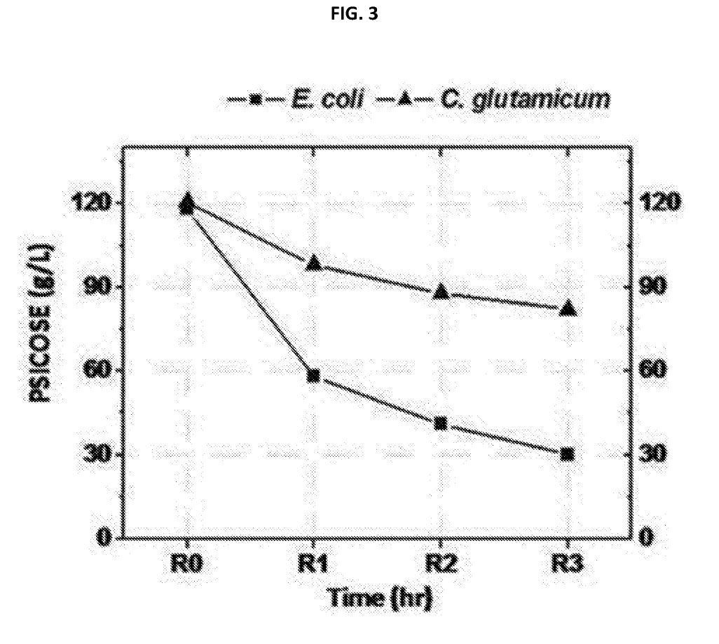

The reaction of converting the reused microorganisms into resting cells were repeated three times at a temperature of 60° C.The first reaction of converting into resting cells is represented by R0, a reaction of converting into resting cells, in which cells recovered from the previous reaction solution are reused once, is represented by R1, a reaction of converting into resting cells, in which the cells are reused twice, is represented by R2, and a reaction of converting into resting cells, in which the cells are reused three times, is represented by R3. Culture conditions and analysis methods were the same as in Example 1. Results are shown in FIG. 3.

Referring to FIG. 3, cells may be reused even when the sugar conversion reaction is performed at a high temperature of 60° C. However, as the cells were reused, enzymatic activity decreased to some extent. In addition, when the cells were reused at high temperature, a Corynebacterium glutamicum strain, a gram-positive bacterium, had higher residual enzymatic activity than an Escherichia coli MG1655 strain, a gram-negative bacterium.

4. Production of D-Psicose from D-Fructose in Various Media for Reaction of Converting into Resting Cells

In Example 1, when a conversion reaction of producing D-psicose from D-fructose in Corynebacterium glutamicum was performed, a minimal medium (1 g K₂HPO₄, 10 g (NH₄)₂SO₄, 0.4 g MgSO₄7H₂O, 20 mg FeSO₄7H₂O, 20 mg MnSO₄5H₂O, 50 mg NaCl, 2 g urea, 0.1 mg biotin, and 0.1 mg thiamine per liter) containing 40% fructose was used. The components of the minimal medium used in this conversion reaction were minimized to prepare a more economical and convenient medium, and the minimal medium was compared with the medium used in Example 1 in terms of D-psicose productivity.

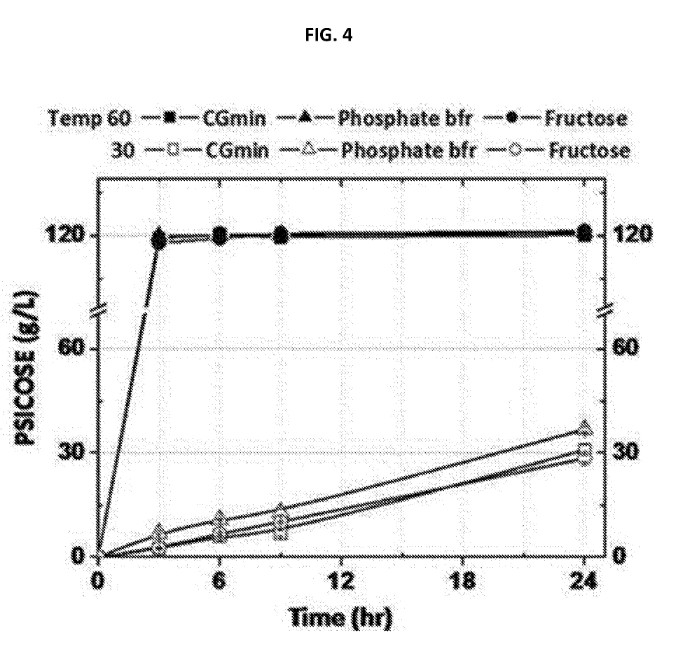

Referring to FIG. 4, even with a phosphate-buffered (pH 7) medium containing 40% fructose and 0.1 mM MnSO₄, more simply, a medium containing only 40% fructose and 0.1 mM MnSO₄, there was no significant difference in the final amount of D-psicose production. The above pattern was observed uniformly at reaction temperatures of 30 and 60° C. Without addition of 0.1 mM MnSO₄, the cofactor of D-psicose-3-epimerase, the production amount decreased.

According to the above example, it can be seen that only D-fructose as a substrate, and MnSO₄, a cofactor of D-psicose-3-epimerase, are required as constituents of a medium used in a conversion reaction for producing D-psicose.

5. Preparation of Recombinant Corynebacterium Glutamicum Containing Polynucleotides Encoding Amino Acid Sequences of D-Psicose 3-Epimerases Derived from Various Strains

The whole genome of Anaerostipes caccae (Anaerostipes caccae DSM 14662; taxid: 411490) was purchased from DSMZ, Germany. The first PCR was performed using a primer pair of SEQ ID NOS: 33 and 34 to include a gene (AP endonuclease; Sequence ID: gb|EDR98778.1|; GI: 167654649; SEQ ID NO: 4), which is presumed to be D-psicose 3-epimerase, using the purchased whole genome as a template. The second PCR was performed using a primer pair of SEQ ID NOS: 35 and 36, which specifically bind to a D-psicose 3-epimerase gene, using the amplified PCR product as a template.

The obtained PCR product was digested with restriction enzymes, BamHI and XbaI, and inserted into the restriction sites of pS208cT-dpe (vector described in Example 1 disclosed in Korean Patent Application No. 10-2013-0060703) digested with the same restriction enzymes to produce a recombinant vector, pS208cT-AcDPE vector.

The resulting pS208cT-AcDPE vector was transformed into wild-type Corynebacterium glutamicum ATCC 13032 and used for the production of D-psicose from D-fructose. A transformation method followed a method specified in Handbook of Corynebacterium glutamicum (Lothar Eggeling et. al., ISBN 0-8493-1821-1, 2005 by CRC press).

The obtained recombinant Corynebacterium glutamicum strain was stored at −80° C. and used for culture.

A plasmid containing a gene (hypothetical protein CLOBOL_00069; Sequence ID: gb|EDP19602.1|; GI:15844190; SEQ ID NO: 9), which is presumed to be D-psicose 3-epimerase of Clostridium bolteae (Clostridium bolteae ATCC BAA-613; taxid:411902), was obtained from Korea Yakult Co., Ltd. PCR was performed using a primer pair of SEQ ID NOS: 37 and 38, which specifically bind to a D-psicose 3-epimerase gene.

The obtained PCR product was digested with restriction enzymes, KpnI and XbaI, and inserted into the restriction sites of pS208cT-dpe (vector described in Example 1 disclosed in Korean Patent Application No. 10-2013-0060703) digested with the same restriction enzymes to produce a recombinant vector, pS208cT-CbDPE vector.

The resulting recombinant pS208cT-CbDPE vector was transformed into wild-type Corynebacterium glutamicum ATCC 13032 using the same method as described above and used for the production of D-psicose from D-fructose. The obtained recombinant Corynebacterium glutamicum strain was stored at −80° C. and used for culture.

The whole genome of Clostridium hylemonae (Clostridium hylemonae DSM 15053; taxid:553973) was purchased from DSMZ, Germany. The first PCR was performed using a primer pair of SEQ ID NOS: 39 and 40 to include a gene (dolichol monophosphate mannose synthase; Sequence ID:ref|WP_006442985.1—; GI:225161759; SEQ ID NO: 10), which is presumed to be D-psicose 3-epimerase, using the purchased whole genome as a template. The second PCR was performed using a primer pair of SEQ ID NOS: 41 and 42, which specifically bind to a D-psicose 3-epimerase gene, using the amplified PCR product as a template.

The obtained PCR product was digested with restriction enzymes, BamHI and XbaI, and inserted into the restriction sites of pS208cT-dpe (vector described in Example 1 disclosed in Korean Patent Application No. 10-2013-0060703) digested with the same restriction enzymes to produce a recombinant vector, pS208cT-ChDPE vector.

The resulting pS208cT-ChDPE vector was transformed into wild-type Corynebacterium glutamicum ATCC 13032 using the same method as described above and used for the production of D-psicose from D-fructose. The obtained recombinant Corynebacterium glutamicum strain was stored at −80° C. and used for culture.

6. Production of D-Psicose from D-Fructose using Recombinant Corynebacterium Glutamicum Strains into Which D-Psicose 3-Epimerases Derived from Various Strains are Introduced

Corynebacterium glutamicum transformants prepared in Example 5 were used to produce D-psicose from high concentration of D-fructose.

The transformants were inoculated into a 2YT medium containing 20 μg/mL of kanamycin and subjected to seed culture at 30° C. and 250 rpm. After culture, the seed culture was inoculated into a 2YT medium containing 20 μg/mL of kanamycin and subjected to main culture. The main culture was performed in a grooved 300 ml Erlenmeyer flask with a 60 ml volume at 30° C. and 180 rpm for 7 hours to induce sufficient cell mass and sufficient expression of proteins.

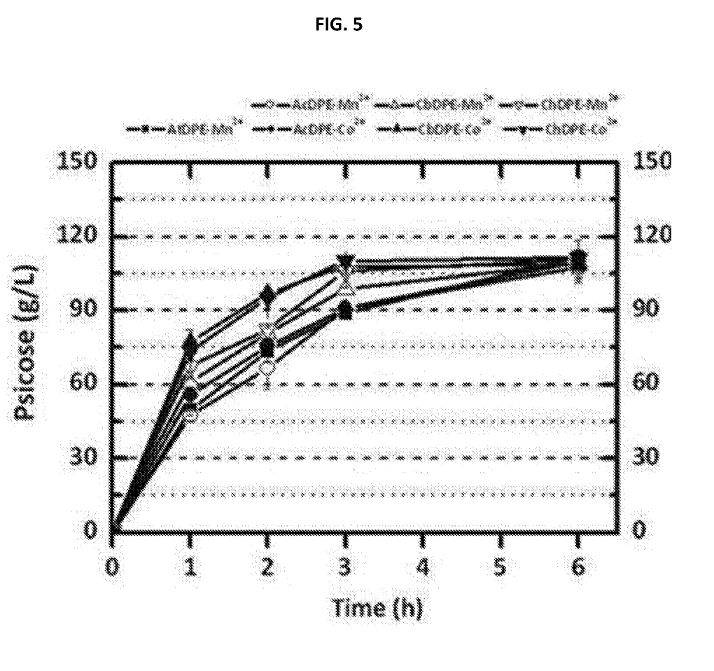

The obtained culture solution was centrifuged to remove a supernatant and recover the microorganisms. The microorganisms were resuspended in a simple conversion reaction medium containing 20 μg/mL kanamycin, 40% (w/v) D-fructose as a substrate, and 0.1 mM concentration of manganese or cobalt known as a primary cofactor of D-psicose 3-epimerase, and then a reaction that converts the microorganisms into resting cells was performed at 55° C. The concentrations of D-fructose and D-psicose were measured in the same method as described in Example 1. The results are shown in FIG. 5 (AtDPE refers to D-psicose 3-epimerase of existing Agrobacterium tumefaciens).

Referring to FIG. 5, when using cobalt rather than manganese as a cofactor, the production rate of D-psicose was slightly faster in all types of D-psicose 3-epimerase-introduced recombinant strains.

In the case of recombinant Corynebacterium glutamicum into which Anaerostipes-derived and Agrobacterium-derived D-psicose 3-epimerases were introduced, respectively, the production amount of D-psicose reached equilibrium at 6 hours. On the other hand, in the case of recombinant Corynebacterium glutamicum into which Clostridium-derived D-psicose 3-epimerase was introduced, the production amount of D-psicose reached equilibrium at 3 hours when manganese was used as a cofactor.

It was confirmed that the Clostridium-derived D-psicose 3-epimerase produced D-psicose from D-fructose, and furthermore, it was found that the production rate of D-psicose was faster than that of Agrobacterium-derived D-psicose 3-epimerase.

7. Continuous Maintenance of D-Psicose Production Activity of Recombinant Corynebacterium Glutamicum Strains at High Temperature, into Which D-Psicose 3-Epimerases Derived from Various Strains are Introduced

In the results of Examples 1 and 2, when a conversion reaction was carried out at a high temperature of 50° C. or higher, all D-psicose was produced within 3 hours. Because of the rapid production of D-psicose from D-fructose at high temperature, a stable D-psicose 3-epimerase is required even at constant high temperature when the cells are reused. Accordingly, it was confirmed to what extent the activity of D-psicose 3-epimerases derived from various strains was maintained at high temperature.

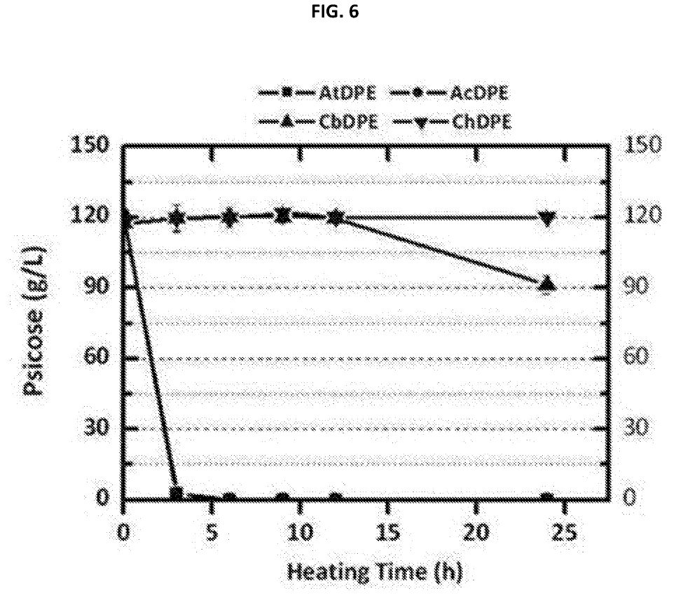

The cells obtained by the method described in Example 1 were suspended in 2YT and the heat of 60° C. was continuously given for 0, 3, 6, 9, 12, and 24 hours using a shaking incubator. The heat-treated cells were collected at each time point and suspended in a simple medium for a conversion reaction containing only 20 μg/mL kanamycin, 0.1 mM manganese, and 40%(w/v) D-fructose, followed by a conversion reaction at 60° C. for 3 hours. The concentrations of D-fructose and D-psicose were measured by the same method as described in Example 1. The results are shown in FIG. 6.

Referring to FIG. 6, in the case of conventional recombinant Corynebacterium glutamicum into which Agrobacterium-derived D-psicose 3-epimerase and Anaerostipes-derived D-psicose 3-epimerase were introduced, respectively, the production of D-psicose hardly occurred when heat of 60° C. was applied for 3 hours. On the other hand, in the case of recombinant Corynebacterium glutamicum into which Clostridium-derived D-psicose 3-epimerase was introduced, the production of D-psicose was maintained even after heat was applied for 24 hours. These results indicate that Clostridium-derived D-psicose 3-epimerase is better than Agrobacterium-derived D-psicose 3-epimerase in the production of D-psicose in a high temperature process.

In the case of Agrobacterium tumefaciens-derived D-psicose 3-epimerase, when the 33rd and 213th amino acids in a sequence (Reference 1) important for thermal stability are substituted with leucine and cysteine respectively, the half-life of the enzyme is increased 3.3-fold and 7.2-fold, respectively, at 50° C. Furthermore, it is known that when both of these amino acids are substituted, the half-life of the enzyme is increased 29.9-fold.

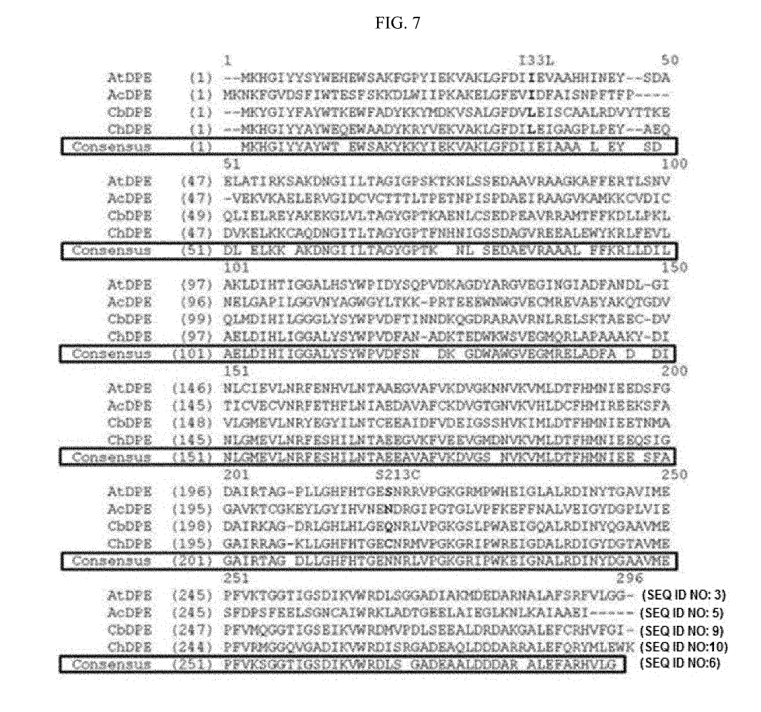

A comparison between the amino acid sequence of Agrobacterium tumefaciens-derived D-psicose 3-epimerase and the amino acid sequence of Clostridium-derived D-psicose 3-epimerase is shown in FIG. 7. As a result, Clostridium-derived D-psicose 3-epimerase with high thermal stability had one or two amino acid sequence characteristics important for thermal stability as described above.

8. Production of D-Psicose from D-Fructose by Cell Recovery and Reuse in Reaction of Converting Recombinant Corynebacterium Glutamicum, into Which Clostridium-Derived D-Psicose 3-Epimerase is Introduced, into Resting Cells

In Example 7, it was confirmed that Clostridium-derived D-psicose 3-epimerase exhibited high stability at high temperature. Therefore, among two Clostridium-derived D-psicose 3-epimerase-introduced recombinant strains, the effect of reusing cells after a conversion reaction at high temperature was confirmed for recombinant Corynebacterium glutamicum into which Clostridium hylemonae-derived D-psicose 3-epimerase was introduced.

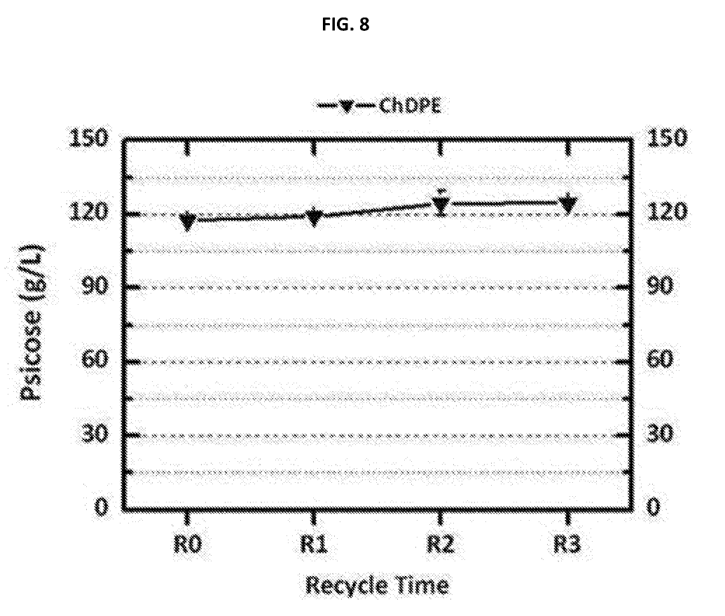

Cells obtained in the same method as described in Example 3 were subjected to a reaction for converting into resting cells at 60° C. for 3 hours, and then the cells were recovered again and reacted in the same manner. The cells were reused three times in total (experiments were carried out under the same conditions as Example 3). The first reaction of converting into resting cells is represented by R0, a reaction of converting into resting cells, in which cells recovered from the previous reaction solution are reused once, is represented by R1, a reaction of converting into resting cells, in which the cells are reused twice, is represented by R2, and a reaction of converting into resting cells, in which the cells are reused three times, is represented by R3. The results are shown in FIG. 8.

Referring to FIG. 8, when cells were reused several times at high temperature, recombinant cells, into which Clostridium-derived D-psicose 3-epimerase was introduced, showed no reduction in the production amount of D-psicose. When the results in Example 7 are taken into consideration, it is confirmed that the thermal stability of an enzyme itself is very advantageous not only in the continuous sugar conversion reaction at high temperature but also in reuse of cells.

1. A method of preparing D-psicose, comprising a step of reacting D-fructose as a substrate and an epimerase thereof in microorganisms at a temperature of 40° C. or higher.

2. The method according to claim 1, wherein the microorganisms express the epimerase endogenously or by transformation.

3. The method according to claim 1, further comprising a step of inducing the microorganisms to have resting cells by culturing the microorganisms in a medium not containing the D-fructose before the reaction.

4. The method according to claim 1, further comprising a step of recovering and reusing the microorganisms to convert D-fructose into D-psicose after the reaction.

5. The method according to claim 4, wherein the microorganisms are gram-positive bacteria.

6. The method according to claim 1, wherein the D-fructose is provided from a medium containing only inorganic salts and D-fructose.

7. The method according to claim 6, wherein the inorganic salts are manganese salts or cobalt salts.

8. The method according to claim 1, wherein the reaction temperature is 40 to 90° C.

9. The method according to claim 1, wherein the microorganisms are Escherichia, Bacillus, Corynebacterium, Actinomyces, yeasts, Kluyveromyces or combinations thereof.

10. The method according to claim 1, wherein the microorganisms are transformed with a gene encoding Agrobacterium tumefaciens-derived D-psicose 3-epimerase corresponding to SEQ ID NO: 1 or a gene encoding Anaerostipes caccae-derived D-psicose 3-epimerase corresponding to SEQ ID NO: 2.

11. The method according to claim 1, wherein the microorganisms are transformed with a gene encoding an amino acid sequence of SEQ ID NO: 5.

12. The method according to claim 1, wherein the microorganisms are transformed with a gene encoding an amino acid sequence in which the 32nd amino acid is substituted with leucine or the 196th amino acid is substituted with cysteine in an amino acid sequence of SEQ ID NO: 6.

13. The method according to claim 1, wherein the microorganisms are transformed with a gene encoding Clostridium-derived D-psicose 3-epimerase.

14. The method according to claim 13, wherein the gene is a gene encoding Clostridium bolteae-derived D-psicose 3-epimerase corresponding to SEQ ID NO: 7 or a gene encoding Clostridium hylemonae-derived D-psicose 3-epimerase corresponding to SEQ ID NO: 8.


SEQUENCE LISTING (42 sequences)

SEQ ID NO: 1 - DNA, 870 bp, Agrobacterium tumefaciens
atgaaacacggcatctattattcttactgggaacatgagtggagcgccaagttcggtccctatatcgagaaggtcgccaagctcggtttcgacatcatcgaagtcgccgcccaccatatcaacgaatacagcgacgccgaactcgcgaccatcaggaagagcgcgaaggataacggcatcatcctcaccgccggcatcggtccgtcgaaaaccaagaacctgtcgtcggaagatgctgcggtgcgtgcggccggcaaggcgttctttgaaagaaccctttcgaacgtcgccaagctcgatatccacaccatcggcggcgcattgcattcctattggccaatcgattattcgcagcccgtcgacaaggcaggcgattatgcgcgcggcgtcgagggtatcaacggcattgccgatttcgccaatgatctcggcatcaacctgtgcatcgaagtcctcaaccgctttgaaaaccacgtcctcaacacggcggcggaaggcgtcgcttttgtgaaggatgtcggcaagaacaatgtgaaagtcatgctggataccttccacatgaacatcgaggaagacagtttcggtgacgccatccgcacggccggcccgcttctggggcacttccataccggtgaaagcaatcgccgcgtaccgggcaagggcagaatgccgtggcacgaaatcggccttgcgctgcgtgatatcaactacaccggcgcggtaatcatggagcctttcgtcaagacaggcggcaccatcggctcggatatcaaggtgtggcgcgacctgagcggtggcgccgacatcgcgaaaatggatgaagatgcccgcaatgcgctggcattctcccgcttcgttcttggtggctaa

SEQ ID NO: 2 - DNA, 858 bp, Anaerostipes caccae
atgaaaaataaattcggagttgacagttttatttggactgaatccttttctaaaaaagatttatggatcatccccaaggcaaaagaactgggatttgaagtcatcgactttgcgatctccaacccattcacattccctgtagagaaagtgaaggcagagctagagagagtgggaatcgactgtgtctgcactaccacgctgacacctgaaaccaatccgatttctccggatgccgagatccgtgcggcaggcgtaaaagccatgaaaaaatgtgtggatatctgcaacgaactgggtgcaccgatcttaggcggtgtaaattatgcaggctggggatatctgacgaagaagccaaggaccgaggaagagtggaactggggcgtagagtgcatgagggaagttgccgagtacgcaaagcaaaccggagatgttaccatctgtgtggaatgtgtcaacagatttgaaacccacttcttaaacattgcggaagatgcagtggccttctgtaaggatgttggaacaggaaatgtcaaggttcatctcgactgcttccatatgatcagagaagaaaagagctttgcaggggcagtaaagacctgcggcaaagaatatctcggatacattcatgtcaatgaaaacgacagaggtattcctggaacagggcttgtaccgtttaaagaatttttcaatgcattagtagagatcgggtatgacggacctttggtgatcgaatcttttgatccgagctttgaagaactgtccggcaactgtgcgatctggagaaaacttgccgatactggagaagaacttgcgattgaagggctgaaaaatctgaaagccatcgctgctgagatataa

SEQ ID NO: 3 - protein, 289 aa, Agrobacterium tumefaciens
MKHGIYYSYWEHEWSAKFGPYIEKVAKLGFDIIEVAAHHINEYSDAELATIRKSAKDNGIILTAGIGPSKTKNLSSEDAAVRAAGKAFFERTLSNVAKLDIHTIGGALHSYWPIDYSQPVDKAGDYARGVEGINGIADFANDLGINLCIEVLNRFENHVLNTAAEGVAFVKDVGKNNVKVMLDTFHMNIEEDSFGDAIRTAGPLLGHFHTGESNRRVPGKGRMPWHEIGLALRDINYTGAVIMEPFVKTGGTIGSDIKVWRDLSGGADIAKMDEDARNALAFSRFVLGG

SEQ ID NO: 4 - protein, 285 aa, Anaerostipes caccae
MKNKFGVDSFIWTESFSKKDLWIIPKAKELGFEVIDFAISNPFTFPVEKVKAELERVGIDCVCTTTLTPETNPISPDAEIRAAGVKAMKKCVDICNELGAPILGGVNYAGWGYLTKKPRTEEEWNWGVECMREVAEYAKQTGDVTICVECVNRFETHFLNIAEDAVAFCKDVGTGNVKVHLDCFHMIREEKSFAGAVKTCGKEYLGYIHVNENDRGIPGTGLVPFKEFFNALVEIGYDGPLVIESFDPSFEELSGNCAIWRKLADTGEELAIEGLKNLKAIAAEI

SEQ ID NO: 5 - protein, 289 aa, Agrobacterium tumefaciens
MKHGIYYSYWEHEWSAKFGPYIEKVAKLGFDILEVAAHHINEYSDAELATIRKSAKDNGIILTAGIGPSKTKNLSSEDAAVRAAGKAFFERTLSNVAKLDIHTIGGALHSYWPIDYSQPVDKAGDYARGVEGINGIADFANDLGINLCIEVLNRFENHVLNTAAEGVAFVKDVGKNNVKVMLDTFHMNIEEDSFGDAIRTAGPLLGHFHTGECNRRVPGKGRMPWHEIGLALRDINYTGAVIMEPFVKTGGTIGSDIKVWRDLSGGADIAKMDEDARNALAFSRFVLGG

SEQ ID NO: 6 - protein, 269 aa, Artificial Sequence
  psicose 3-epimerase consensus
MKHGIYYAYWTEWSAKYKKYIEKVAKLGFDIIEIAAALEYSDDLELKKAKDNGIILTAGYGPTKNLSEDAEVRAAALFFKRLLDILAELDIHIIGGALYSYWPVDFSNDKGDWAWGVEGMRELADFADDINLGMEVLNRFESHILNTAEEAVAFVKDVGSNVKVMLDTFHMNIEESFAGAIRTAGDLLGHFHTGENNRLVPGKGRIPWKEIGNALRDINYDGAAVMEPFVKSGGTIGSDIKVWRDLSGADEAALDDDARALEFARHVLG

SEQ ID NO: 7 - DNA, 876 bp, Clostridium bolteae
atgaaatatggtatttattttgcttattggacgaaggaatggtttgctgattataagaagtatatggataaggtgtctgctttggggtttgatgtcctggaaatttcctgtgcagccctcagagatgtatacacaaccaaggaacagctgattgagctacgtgaatacgccaaagaaaaagggcttgttctaacagctggttatggacctactaaagcagaaaatctgtgttcagaagacccggaggcagtgagacgggccatgacattcttcaaggacctgcttccaaagctgcagttaatggatatccatatcctgggagggggattatattcctactggcccgtggattttaccattaataatgacaagcagggagaccgggccagggctgtcaggaatctgagggaattgtccaaaacagcggaggaatgtgacgtggtgcttggaatggaggtactgaaccgctatgaggggtatattcttaatacctgtgaagaggcaattgattttgtcgatgagattggaagcagccatgtaaaaatcatgctggatactttccatatgaatattgaagagacaaatatggctgatgcaatccgcaaggcgggagacaggctgggacatctccatctgggagaacagaaccgcctggtgccgggaaaaggcagcctgccatgggctgagatagggcaggcgctccgtgatattaactatcagggagccgctgtcatggaaccttttgtcatgcagggagggaccatcggttctgagataaaggtatggagagacatggtgccggatctttctgaggaagcactggacagggatgcaaagggtgcgctggaattctgcaggcatgtgtttggtatctaa

SEQ ID NO: 8 - DNA, 870 bp, Clostridium hylemonae
atgaaacatggtatctattatgcatactgggaacaagaatgggcggccgactacaagcgctatgttgaaaaggtggcaaagcttgggtttgacattctggagatcggcgctgggccgctgccggaatacgcagagcaggatgtgaaggaactgaagaaatgtgcgcaggacaatgggatcacgctgacggccggatatggtccgacgttcaaccacaatatcggttcttcagacgccggggtaagggaagaggcgctggaatggtataagaggttatttgaagtgctggcagagcttgatatccacctgatcggaggggcgctctattcttactggcctgtcgattttgcaaacgccgataaaacggaagactggaagtggagtgtagagggcatgcagaggctggcgccggccgcggccaaatatgacatcaacctgggcatggaagttctgaaccggtttgagagccatatcctgaatacagccgaggaaggtgtgaagtttgtagaggaagtcggcatggacaacgtaaaggtcatgctggatacattccatatgaatatagaagagcaaagcataggcggcgcgatccgccgggcaggaaaactgctcgggcatttccacaccggagaatgcaaccgcatggtgcccgggaagggacgtattccatggcgtgagataggggatgctctccgtgatatcggatatgacggaactgctgtaatggagccgttcgttcgcatgggaggacaggtcggcgctgatatcaaggtgtggagagacataagccgtggagcagacgaggcacagcttgacgatgacgcgcgccgtgcgctggagttccagagatatatgctggagtggaagtaa

SEQ ID NO: 9 - protein, 291 aa, Clostridium bolteae
MKYGIYFAYWTKEWFADYKKYMDKVSALGFDVLEISCAALRDVYTTKEQLIELREYAKEKGLVLTAGYGPTKAENLCSEDPEAVRRAMTFFKDLLPKLQLMDIHILGGGLYSYWPVDFTINNDKQGDRARAVRNLRELSKTAEECDVVLGMEVLNRYEGYILNTCEEAIDFVDEIGSSHVKIMLDTFHMNIEETNMADAIRKAGDRLGHLHLGEQNRLVPGKGSLPWAEIGQALRDINYQGAAVMEPFVMQGGTIGSEIKVWRDMVPDLSEEALDRDAKGALEFCRHVFGI

SEQ ID NO: 10 - protein, 289 aa, Clostridium hylemonae
MKHGIYYAYWEQEWAADYKRYVEKVAKLGFDILEIGAGPLPEYAEQDVKELKKCAQDNGITLTAGYGPTFNHNIGSSDAGVREEALEWYKRLFEVLAELDIHLIGGALYSYWPVDFANADKTEDWKWSVEGMQRLAPAAAKYDINLGMEVLNRFESHILNTAEEGVKFVEEVGMDNVKVMLDTFHMNIEEQSIGGAIRRAGKLLGHFHTGECNRMVPGKGRIPWREIGDALRDIGYDGTAVMEPFVRMGGQVGADIKVWRDISRGADEAQLDDDARRALEFQRYMLEWK

SEQ ID NO: 11 - DNA, 963 bp, Escherichia coli
atgattaagaaaatcggtgtgttgacaagcggcggtgatgcgccaggcatgaacgccgcaattcgcggggttgttcgttctgcgctgacagaaggtctggaagtaatgggtatttatgacggctatctgggtctgtatgaagaccgtatggtacagctagaccgttacagcgtgtctgacatgatcaaccgtggcggtacgttcctcggttctgcgcgtttcccggaattccgcgacgagaacatccgcgccgtggctatcgaaaacctgaaaaaacgtggtatcgacgcgctggtggttatcggcggtgacggttcctacatgggtgcaatgcgtctgaccgaaatgggcttcccgtgcatcggtctgccgggcactatcgacaacgacatcaaaggcactgactacactatcggtttcttcactgcgctgagcaccgttgtagaagcgatcgaccgtctgcgtgacacctcttcttctcaccagcgtatttccgtggtggaagtgatgggccgttattgtggagatctgacgttggctgcggccattgccggtggctgtgaattcgttgtggttccggaagttgaattcagccgtgaagacctggtaaacgaaatcaaagcgggtatcgcgaaaggtaaaaaacacgcgatcgtggcgattaccgaacatatgtgtgatgttgacgaactggcgcatttcatcgagaaagaaaccggtcgtgaaacccgcgcaactgtgctgggccacatccagcgcggtggttctccggtgccttacgaccgtattctggcttcccgtatgggcgcttacgctatcgatctgctgctggcaggttacggcggtcgttgtgtaggtatccagaacgaacagctggttcaccacgacatcatcgacgctatcgaaaacatgaagcgtccgttcaaaggtgactggctggactgcgcgaaaaaactgtattaa

SEQ ID NO: 12 - protein, 320 aa, Escherichia coli
MIKKIGVLTSGGDAPGMNAAIRGVVRSALTEGLEVMGIYDGYLGLYEDRMVQLDRYSVSDMINRGGTFLGSARFPEFRDENIRAVAIENLKKRGIDALVVIGGDGSYMGAMRLTEMGFPCIGLPGTIDNDIKGTDYTIGFFTALSTVVEAIDRLRDTSSSHQRISVVEVMGRYCGDLTLAAAIAGGCEFVVVPEVEFSREDLVNEIKAGIAKGKKHAIVAITEHMCDVDELAHFIEKETGRETRATVLGHIQRGGSPVPYDRILASRMGAYAIDLLLAGYGGRCVGIQNEQLVHHDIIDAIENMKRPFKGDWLDCAKKLY

SEQ ID NO: 13 - DNA, 450 bp, Escherichia coli
atgaaaaagattgcatttggctgtgatcatgtcggtttcattttaaaacatgaaatagtggcacatttagttgagcgtggcgttgaagtgattgataaaggaacctggtcgtcagagcgtactgattatccacattacgccagtcaagtcgcactggctgttgctggcggagaggttgatggcgggattttgatttgtggtactggcgtcggtatttcgatagcggcgaacaagtttgccggaattcgcgcggtcgtctgtagcgaaccttattccgcgcaactttcgcggcagcataacgacaccaacgtgctggcttttggttcacgagtggttggcctcgaactggcaaaaatgattgtggatgcgtggctgggcgcacagtacgaaggcggtcgtcatcaacaacgcgtggaggcgattacggcaatagagcagcggagaaattga

SEQ ID NO: 14 - DNA, 891 bp, Escherichia coli
atgagccagtcagagtttgattcagcgcttccgaacggtatagggttagcgccttacctgcgaatgaagcaggaaggaatgacagaaaatgaaagccgcatcgtggagtggttactcaaacccggtaacctgagttgtgcacccgcaattaaagatgtcgcagaagctctggcggtatctgaagcgatgatagttaaggtatcaaagctgctggggtttagcggctttcgtaacttacgcagtgcgctggaagattatttttctcagtcagaacaggtattgccttccgagttggcttttgatgaagcgccgcaggatgtggtgaataaggtatttaacatcactttacgcaccattatggaaggtcagtcgatcgtcaacgttgatgagatccaccgtgccgcccgctttttctatcaggccagacagcgggatttgtacggtgccggaggatcaaatgctatctgtgctgatgtacagcacaagttcttgcgcattggcgtacgctgtcaggcctatcctgatgctcacatcatgatgatgtccgcttcgttgttacaggaaggagatgttgtgctggtagtgacccattccgggcgaaccagtgatgtaaaagcggccgtagaactggcaaaaaagaacggggcaaagattatttgtataacccatagctaccattcaccgatagcgaaactggccgattatattatttgctcaccagccccggaaacgccgttattaggtcgtaatgcctcggcaagaatattacaactaactttgctggacgctttttttgtctctgtcgcccagctcaacattgaacaagctaatattaatatgcaaaaaaccggcgcaattgttgatttcttctcaccaggcgcgctgaaataa

SEQ ID NO: 15 - DNA, 936 bp, Escherichia coli
atgaataaatatctgaaatatttcagcggcacactcgtgggcttaatgttgtcaaccagcgcttttgctgccgccgaatatgctgtcgtattgaaaaccctctccaacccattttgggtagatatgaaaaaaggcattgaagatgaagcaaaaacactgggcgtcagcgttgatatttttgcctctccttcagaaggcgattttcaatctcaattgcagttatttgaagatctcagtaataaaaattacaaaggtatcgccttcgctccattatcctcagtgaatctggtcatgcctgtcgcccgcgcatggaaaaaaggcatttatctggttaatctcgatgaaaaaatcgacatggataatctgaaaaaagctggcggcaatgtggaagcttttgtcaccaccgataacgttgctgtcggggcgaaaggcgcgtcgttcattattgacaaattgggcgctgaaggtggtgaagtcgcaatcattgagggtaaagccggtaacgcctccggtgaagcgcgtcgtaatggtgccaccgaagccttcaaaaaagcaagccagatcaagcttgtcgccagccagcctgccgactgggaccgcattaaagcactggatgtcgccactaacgtgttgcaacgtaatccgaatattaaagcgatctattgcgcgaatgacacgatggcaatgggtgttgctcaggcagtcgcaaacgccggaaaaacgggaaaagtgctggtcgtcggtacagatggcattccggaagcccgcaaaatggtggaagccggacaaatgaccgcgacggttgcccagaacccggcggatatcggcgcaacgggtctgaagctgatggttgacgctgagaaatccggcaaggttatcccgctggataaagcaccggaatttaaactggtcgattcaatcctggtcactcaataa

SEQ ID NO: 16 - DNA, 1533 bp, Escherichia coli
atggccacgccatatatatcgatggcggggatcggcaagtcctttggtccggttcacgcattaaagtcggttaatttaacggtttatcctggtgaaatacatgcattactaggagaaaatggcgcgggtaaatccacgctaatgaaagttttatccggaatacatgagccgaccaaaggcaccattaccattaataacattagctataacaagctggatcataaattagcggcacaactcggtatcgggattatttatcaggaactcagcgttattgatgaattaaccgtactggaaaatttatatattggtcgtcatctgacgaaaaaaatctgtggcgtcaatattatcgactggcgagaaatgcgtgtccgcgccgccatgatgttattacgcgtgggcttgaaagttgatctagatgagaaagtggcgaatttatctatcagccacaagcagatgctagaaattgccaaaacgctgatgctcgatgccaaagtcatcatcatggatgaacccacctcctcactcaccaataaagaggtggactatctgtttctgatcatgaatcagttgcgtaaagagggtacggccatcgtctatatctcgcataagttggcggaaattcgccgtatttgcgaccgctatacggtgatgaaagacggcagcagcgtttgcagcggcatagtaagcgatgtgtcaaatgacgatatcgtccgtctgatggtaggccgcgaactgcaaaaccgttttaacgcgatgaaggagaatgtcagcaaccttgcgcacgaaacggtttttgaggtgcggaacgtcaccagtcgtgacagaaaaaaggtccgggatatctcatttagcgtctgccggggagaaatattaggctttgccggactggtcggttccggacgtactgaactgatgaattgtctgtttggcgtggataaacgcgctggcggagaaatccgtcttaatggcaaagatatctctccacgttcacccctggatgccgtgaaaaaagggatggcttacatcactgaaagccgccgggataacggttttttccccaacttttccatcgctcagaacatggcgatcagccgcagtctgaaagacggcggctataaaggcgcgatgggcttgtttcatgaagttgacgagcaacgtaccgctgaaaatcaacgcgaactgctggcgctgaaatgtcattcggtaaaccagaatatcaccgaactctccgggggaaatcagcagaaagtcctgatctccaaatggctgtgctgttgcccggaagtgattattttcgatgaacctacccgcggcatcgacgttggcgcgaaagccgaaatttacaaagtgatgcgccaactggcggacgacggaaaagtcatcctgatggtgtcatctgaactacctgaaattatcaccgtctgcgaccgcatcgccgtgttctgcgaaggacgactgacgcaaatcctgacgaatcgcgatgacatgagcgaagaggagattatggcatgggctttaccacaagagtaa

SEQ ID NO: 17 - DNA, 981 bp, Escherichia coli
atgggctttaccacaagagtaaaaagcgaagcgagcgagaagaaaccgttcaactttgcgctgttctgggataaatacggcaccttttttatcctggcgatcatcgtcgccatctttggttcgctgtcaccagaatattttctgaccaccaataatattacccagatttttgttcaaagctccgtgacggtattgatcggcatgggcgagtttttcgctatcctggtcgctggtatcgacctctcggttggcgcgattctggcgctttccggtatggtgaccgccaaactgatgttggcaggtgttgacccgtttctcgcagcgatgattggcggtgtactggttggcggcgcactgggggcgatcaacggctgcctggtcaactggacggggctacacccgttcatcatcacccttggcaccaacgcgattttccgtgggatcacgctggtgatctccgatgccaactcggtatacggcttctcatttgacttcgtgaacttctttgccgccagcgtaattgggatacctgtccccgttatcttctcactaattgtcgcgctcatcctttggtttctgacaacgcgtatgcggctcgggcgcaacatctacgcactgggcggcaacaaaaattcggcgttctattccgggattgacgtgaaattccacatcctggtggtgtttatcatctccggtgtttgtgcaggtctggcaggcgtcgtctcaactgcacgactcggtgccgcagaaccgcttgccggtatgggttttgaaacctatgccattgccagcgccatcattggcggcaccagtttcttcggcggcaaggggcgcattttctctgtggtgattggcgggttgatcatcggcaccatcaacaacggtctgaatattttgcaggtacaaacctattaccaactggtggtgatgggcggattaattatcgcggctgtcgcccttgaccgtcttatcagtaagtaa

SEQ ID NO: 18 - DNA, 696 bp, Escherichia coli
atgaaaatctccccctcgttaatgtgtatggatctgctgaaatttaaagaacagatcgaatttatcgacagccatgccgattacttccacatcgatatcatggacggtcactttgtccccaatctgacactctcaccgttcttcgtaagtcaggttaaaaaactggcaactaaaccgctcgactgtcatctgatggtgacgcggccgcaggattacattgctcaactggcgcgtgcgggagcagatttcatcactctgcatccggaaaccatcaacggccaggcgttccgcctgattgatgaaatccgccgtcatgacatgaaagtggggctgatccttaacccggagacgccagttgaggccatgaaatactatatccataaggccgataaaattacggtcatgactgtcgatcccggctttgccggacaaccgttcattcctgaaatgctggataaacttgccgaactgaaggcatggcgtgaacgagaaggtctggagtacgaaattgaggtggacggttcctgcaaccaggcaacttacgaaaaactgatggcggcaggggcggatgtctttatcgtcggcacttccggcctgtttaatcatgcggaaaatatcgacgaagcatggagaattatgaccgcgcagattctggctgcaaaaagcgaggtacagcctcatgcaaaaacagcataa

SEQ ID NO: 19 - DNA, 930 bp, Escherichia coli
atgcaaaaacagcataacgtcgtagcgggcgtggatatgggggcaacgcatatccgcttttgtctgcggacagcagaaggtgaaacgctacactgcgaaaaaaagcggaccgcagaagtcattgctcccggcctggtgtcgggtatcggcgaaatgattgacgagcaactcaggcgctttaacgctcgctgtcatggtctggtgatgggatttccggcgctggtcagtaaagataaacgcaccattatttctacgcctaacctgccgttaacagcggcggatttatatgatctcgccgataagctcgaaaatacgctgaattgtccggttgagttttcccgcgacgttaacctgcaactctcctgggacgtagtagaaaaccgccttacgcaacaactggttctggcggcctatctcggtacggggatggggttcgcagtgtggatgaacggtgcgccgtggacgggtgcacacggtgtggcaggcgaactgggtcatatccccctgggagatatgacccaacactgcgcgtgtggcaatcctgggtgcctggaaaccaattgctctggaatggcgctaagacgctggtacgaacaacagccccgaaattacccattgcgcgatcttttcgtccatgcggaaaacgcccctttcgtccagagtctgcttgaaaacgcggcacgggccattgccaccagcattaatctgttcgatcccgatgcggtgatcctgggcggtggcgtgatggatatgcccgccttcccacgcgagactctcgttgccatgacccaaaagtacctgcgccgtccactgccgcatcaggtcgtgcgctttattgccgcctcatcttctgactttaatggcgctcagggtgcagcaatattggcgcatcaacgttttttgccacagttctgtgctaaagccccatga

SEQ ID NO: 20 - protein, 149 aa, Escherichia coli
MKKIAFGCDHVGFILKHEIVAHLVERGVEVIDKGTWSSERTDYPHYASQVALAVAGGEVDGGILICGTGVGISIAANKFAGIRAVVCSEPYSAQLSRQHNDTNVLAFGSRVVGLELAKMIVDAWLGAQYEGGRHQQRVEAITAIEQRRN

SEQ ID NO: 21 - protein, 296 aa, Escherichia coli
MSQSEFDSALPNGIGLAPYLRMKQEGMTENESRIVEWLLKPGNLSCAPAIKDVAEALAVSEAMIVKVSKLLGFSGFRNLRSALEDYFSQSEQVLPSELAFDEAPQDVVNKVFNITLRTIMEGQSIVNVDEIHRAARFFYQARQRDLYGAGGSNAICADVQHKFLRIGVRCQAYPDAHIMMMSASLLQEGDVVLVVTHSGRTSDVKAAVELAKKNGAKIICITHSYHSPIAKLADYIICSPAPETPLLGRNASARILQLTLLDAFFVSVAQLNIEQANINMQKTGAIVDFFSPGALK

SEQ ID NO: 22 - protein, 311 aa, Escherichia coli
MNKYLKYFSGTLVGLMLSTSAFAAAEYAVVLKTLSNPFWVDMKKGIEDEAKTLGVSVDIFASPSEGDFQSQLQLFEDLSNKNYKGIAFAPLSSVNLVMPVARAWKKGIYLVNLDEKIDMDNLKKAGGNVEAFVTTDNVAVGAKGASFIIDKLGAEGGEVAIIEGKAGNASGEARRNGATEAFKKASQIKLVASQPADWDRIKALDVATNVLQRNPNIKAIYCANDTMAMGVAQAVANAGKTGKVLVVGTDGIPEARKMVEAGQMTATVAQNPADIGATGLKLMVDAEKSGKVIPLDKAPEFKLVDSILVTQ

SEQ ID NO: 23 - protein, 326 aa, Escherichia coli
MGFTTRVKSEASEKKPFNFALFWDKYGTFFILAIIVAIFGSLSPEYFLTTNNITQIFVQSSVTVLIGMGEFFAILVAGIDLSVGAILALSGMVTAKLMLAGVDPFLAAMIGGVLVGGALGAINGCLVNWTGLHPFIITLGTNAIFRGITLVISDANSVYGFSFDFVNFFAASVIGIPVPVIFSLIVALILWFLTTRMRLGRNIYALGGNKNSAFYSGIDVKFHILVVFIISGVCAGLAGVVSTARLGAAEPLAGMGFETYAIASAIIGGTSFFGGKGRIFSVVIGGLIIGTINNGLNILQVQTYYQLVVMGGLIIAAVALDRLISK

SEQ ID NO: 24 - protein, 326 aa, Escherichia coli; identical to SEQ ID NO: 23

SEQ ID NO: 25 - protein, 309 aa, Escherichia coli
MQKQHNVVAGVDMGATHIRFCLRTAEGETLHCEKKRTAEVIAPGLVSGIGEMIDEQLRRFNARCHGLVMGFPALVSKDKRTIISTPNLPLTAADLYDLADKLENTLNCPVEFSRDVNLQLSWDVVENRLTQQLVLAAYLGTGMGFAVWMNGAPWTGAHGVAGELGHIPLGDMTQHCACGNPGCLETNCSGMALRRWYEQQPRNYPLRDLFVHAENAPFVQSLLENAARAIATSINLFDPDAVILGGGVMDMPAFPRETLVAMTQKYLRRPLPHQVVRFIAASSSDFNGAQGAAILAHQRFLPQFCAKAP

SEQ ID NO: 26 - protein, 309 aa, Escherichia coli; identical to SEQ ID NO: 25

SEQ ID NO: 27 - DNA, 2067 bp, corynebacterium
atgaatagcgtaaataattcctcgcttgtccggctggatgtcgatttcggcgactccaccacggatgtcatcaacaaccttgccactgttattttcgacgctggccgagcttcctccgccgacgcccttgccaaagacgcgctggatcgtgaagcaaagtccggcaccggcgttcctggtcaagttgctatcccccactgccgttccgaagccgtatctgtccctaccttgggctttgctcgcctgagcaagggtgtggacttcagcggacctgatggcgatgccaacttggtgttcctcattgcagcacctgctggcggcggcaaagagcacctgaagatcctgtccaagcttgctcgctccttggtgaagaaggatttcatcaaggctctgcaggaagccaccaccgagcaggaaatcgtcgacgttgtcgatgccgtgctcaacccagcaccaaaaaccaccgagccagctgcagctccggctgcggcggcggttgctgagagtggggcggcgtcgacaagcgttactcgtatcgtggcaatcaccgcatgcccaaccggtatcgcacacacctacatggctgcggattccctgacgcaaaacgcggaaggccgcgatgatgtggaactcgttgtggagactcagggctcttccgctgtcaccccagtcgatccgaagatcatcgaagctgccgacgccgtcatcttcgccaccgacgtgggagttaaagaccgcgagcgtttcgctggcaagccagtcattgaatccggcgtcaagcgcgcgatcaatgagccagccaagatgatcgacgaggccatcgcagcctccaagaacccaaacgcccgcaaggtttccggttccggtgtcgcggcatctgctgaaaccaccggcgagaagctcggctggggcaagcgcatccagcaggcagtcatgaccggcgtgtcctacatggttccattcgtagctgccggcggcctcctgttggctctcggcttcgcattcggtggatacgacatggcgaacggctggcaagcaatcgccacccagttctctctgaccaacctgccaggcaacaccgtcgatgttgacggcgtggccatgaccttcgagcgttcaggcttcctgttgtacttcggcgcagtcctgttcgccaccggccaagcagccatgggcttcatcgtggcagccctgtctggctacaccgcatacgcacttgctggacgcccaggcatcgcgccgggcttcgtcggtggcgccatctccgtcaccatcggcgctggcttcattggtggtctggttaccggtatcttggctggtctcattgccctgtggattggctcctggaaggtgccacgcgtggtgcagtcactgatgcctgtggtcatcatcccgctacttacctcagtggttgttggtctcgtcatgtacctcctgctgggtcgcccactcgcatccatcatgactggtttgcaggactggctatcgtcaatgtccggaagctccgccatcttgctgggtatcatcttgggcctcatgatgtgtttcgacctcggcggaccagtaaacaaggcagcctacctctttggtaccgcaggcctgtctaccggcgaccaagcttccatggaaatcatggccgcgatcatggcagctggcatggtcccaccaatcgcgttgtccattgctaccctgctgcgcaagaagctgttcaccccagcagagcaagaaaacggcaagtcttcctggctgcttggcctggcattcgtctccgaaggtgccatcccattcgccgcagctgacccattccgtgtgatcccagcaatgatggctggcggtgcaaccactggtgcaatctccatggcactgggcgtcggctctcgggctccacacggcggtatcttcgtggtctgggcaatcgaaccatggtggggctggctcatcgcacttgcagcaggcaccatcgtgtccaccatcgttgtcatcgcactgaagcagttctggccaaacaaggccgtcgctgcagaagtcgcgaagcaagaagcacaacaagcagctgtaaacgcataa

SEQ ID NO: 28 - protein, 688 aa, corynebacterium
MNSVNNSSLVRLDVDFGDSTTDVINNLATVIFDAGRASSADALAKDALDREAKSGTGVPGQVAIPHCRSEAVSVPTLGFARLSKGVDFSGPDGDANLVFLIAAPAGGGKEHLKILSKLARSLVKKDFIKALQEATTEQEIVDVVDAVLNPAPKTTEPAAAPAAAAVAESGAASTSVTRIVAITACPTGIAHTYMAADSLTQNAEGRDDVELVVETQGSSAVTPVDPKIIEAADAVIFATDVGVKDRERFAGKPVIESGVKRAINEPAKMIDEAIAASKNPNARKVSGSGVAASAETTGEKLGWGKRIQQAVMTGVSYMVPFVAAGGLLLALGFAFGGYDMANGWQAIATQFSLTNLPGNTVDVDGVAMTFERSGFLLYFGAVLFATGQAAMGFIVAALSGYTAYALAGRPGIAPGFVGGAISVTIGAGFIGGLVTGILAGLIALWIGSWKVPRVVQSLMPVVIIPLLTSVVVGLVMYLLLGRPLASIMTGLQDWLSSMSGSSAILLGIILGLMMCFDLGGPVNKAAYLFGTAGLSTGDQASMEIMAAIMAAGMVPPIALSIATLLRKKLFTPAEQENGKSSWLLGLAFVSEGAIPFAAADPFRVIPAMMAGGATTGAISMALGVGSRAPHGGIFVVWAIEPWWGWLIALAAGTIVSTIVVIALKQFWPNKAVAAEVAKQEAQQAAVNA

SEQ ID NO: 29 - DNA, 1512 bp, corynebacterium
atgaacaccccactccagctcaacactgaaaacctgcaggaaatcgcttcgacttccggagtgcagatcccagcgttcaaccgcgctgacgtcgccccgggcattgtccacttcggtgttggcggattccatcgcgctcaccaagcgatgtacctcaatgaattgatgaatgagggcaaggccttggattggggcatcatcggcatgggtgtcatgccttccgatgtgcgcatgcgcgatgccctggccagccaagatcacctttataccctgaccactaaagctcctgatggaactcttgatcaaaaaatcatcggatccatcattgactacgtgttcgctcccgaggacccagcacgggccgttgcaaccctcgcgcaggactccatccgcattgtttccctcacggtgactgaaggcggatacaacatcgatccggcgacagaagatttcgaccacaccaaccctcgaatcgttgctgaccgcgaagccctgcaggcgggcgatacttccactttgcagaccttctttgggttgatcactgccgcattgatttcccgaaaagaatcaggatctacgccatttaccatcatgagctgcgataacatccaaggcaacggcgatctggctaagcgtttcttcctcgccttcgcacattccgtgtcttctgagctcggcgaatgggtggaaaacaacgtggccttccccaactccatggtggaccgcatcacccctgaaaccaccgacggcgaccgcgatgacatcaaggaaatcggctacatcgatgcgtggccagtggtttctgaagatttcacccaatgggtcctcgaggatgccttcacccagggccgccccgcgtacgaggaggttggcgtgcaggtcgtctccgacgtggagccttatgaattaatgaagctgcgcctgctcaacgcctcccaccagggactttgctacttcggccacttggctggccaccacatggtccacgacgtcatggcggatacccgcttccaggatttcctcctggcttacatggagcgcgaagccacccctaccctcaaggaacttccaggtgtcgatctagatgcttatcgacgccaactcatcgcgcgattcggcaacgccgcagtcaaagacaccgtaccgcgcctgtgtgcggaatcctccgaccgcattccaaagtggctgttgccagtcgtacgcgaaaacctcgcagcaggccgcgacgtcacactttctgcagccatcgtcgcatcctgggcgcgctacgcagaaggcaccgacgagcagggcaacccaataaagattgttgaccgtttgagtgagcgcgtccaagaaaacgcatcaggaaatcgcaccgatattttgtcattcatccgcgaccgtggaatcttcggagacttggtcgatgctgaaccattcaccaaggcatactccgagacactgtcctcccttcatgaccgtggcgcggaagcaaccatcgatgcacttcttacgcaggtaactgtctaa

SEQ ID NO: 30 - protein, 503 aa, corynebacterium
MNTPLQLNTENLQEIASTSGVQIPAFNRADVAPGIVHFGVGGFHRAHQAMYLNELMNEGKALDWGIIGMGVMPSDVRMRDALASQDHLYTLTTKAPDGTLDQKIIGSIIDYVFAPEDPARAVATLAQDSIRIVSLTVTEGGYNIDPATEDFDHTNPRIVADREALQAGDTSTLQTFFGLITAALISRKESGSTPFTIMSCDNIQGNGDLAKRFFLAFAHSVSSELGEWVENNVAFPNSMVDRITPETTDGDRDDIKEIGYIDAWPVVSEDFTQWVLEDAFTQGRPAYEEVGVQVVSDVEPYELMKLRLLNASHQGLCYFGHLAGHHMVHDVMADTRFQDFLLAYMEREATPTLKELPGVDLDAYRRQLIARFGNAAVKDTVPRLCAESSDRIPKWLLPVVRENLAAGRDVTLSAAIVASWARYAEGTDEQGNPIKIVDRLSERVQENASGNRTDILSFIRDRGIFGDLVDAEPFTKAYSETLSSLHDRGAEATIDALLTQVTV

SEQ ID NO: 31 - DNA, 53 bp, Artificial Sequence
  primer
gcaggtaccaggaggtaataaatatgaaacacggcatctattattcttactgg

SEQ ID NO: 32 - DNA, 32 bp, Artificial Sequence
  primer
gctggatccttagccaccaagaacgaagcggg

SEQ ID NO: 33 - DNA, 23 bp, Artificial Sequence
  primer
ccacggaactttcggctgttttg

SEQ ID NO: 34 - DNA, 22 bp, Artificial Sequence
  primer
cttccgtaggtgccacaatatc

SEQ ID NO: 35 - DNA, 44 bp, Artificial Sequence
  primer
cgaggatccaggaggtaataatatgaaaaataaattcggagttg

SEQ ID NO: 36 - DNA, 28 bp, Artificial Sequence
  primer
gtctagattatatctcagcagcgatggc

SEQ ID NO: 37 - DNA, 53 bp, Artificial Sequence
  primer
cggtaccaggaggtaataatatgaaatatggtatttattttgcttattggacg

SEQ ID NO: 38 - DNA, 32 bp, Artificial Sequence
  primer
ctctagattagataccaaacacatgcctgcag

SEQ ID NO: 39 - DNA, 24 bp, Artificial Sequence
  primer
cggactatctgaacgaactgacgg

SEQ ID NO: 40 - DNA, 23 bp, Artificial Sequence
  primer
ctccataagcatcctcctcatcc

SEQ ID NO: 41 - DNA, 45 bp, Artificial Sequence
  primer
cgaggatccaggaggtaataatatgaaacatggtatctattatgc

SEQ ID NO: 42 - DNA, 27 bp, Artificial Sequence
  primer
gtctagattacttccactccagcatat